(19) United States
(12) Patent Application Publication
(10) Pub. No.: US 2024/0033473 A1
(43) Pub. Date: Feb. 1, 2024

(54) SLOTTED LINERS FOR MEDICAL DEVICE

(71) Applicants: Brian B. MARTIN, Santa Cruz, CA (US); Jason MILLER, Campbell, CA (US)

(72) Inventors: Brian B. MARTIN, Santa Cruz, CA (US); Jason MILLER, Campbell, CA (US)

(21) Appl. No.: 18/361,237

(22) Filed: Jul. 28, 2023

Related U.S. Application Data
(60) Provisional application No. 63/369,833, filed on Jul. 29, 2022.

(51) Int. Cl. A61M 25/00

(52) U.S. Cl. CPC A61M 25/0045; A61M 25/0054; A61M 2025/0004

(57) ABSTRACT

Polymeric inner liner, for use with catheters or other medical devices, where the polymeric inner liner tubing can have regions of customized properties created by slot type patterns in the inner liner.

### CROSS-REFERENCE TO RELATED APPLICATIONS

This application is a non-provisional application of U.S. Provisional application 63/369,833 filed on Jul. 29, 2022, the entirety of which is incorporated by reference.

### FIELD OF THE INVENTION

Polymeric inner liners for use with catheters or other medical devices, where the polymeric inner liner tubing can have regions of customized properties created by slot type patterns in the liner.

### BACKGROUND OF THE INVENTION

Medical catheters allow physicians to apply a variety of different therapies within the body of a patient. Many catheters access remote regions of the human body for delivering diagnostic or therapeutic tools and/or agents to those sites. Alternatively, the catheter can comprise a shaft or support for a therapeutic working end (e.g., balloon, filter retriever, electrode, etc.). Some catheters, including but not limited to catheters for neurovascular use, are intended to be advanced from a main artery (e.g., a femoral or radial artery) through tortuous anatomy into a small cerebral vessel. As such, the catheter must be configured with varying structural traits due to the varying regions of the anatomy through which the catheter passes. Many times, the vascular pathways wind back upon themselves in a multi-looped path making it difficult for catheter design to meet the requirements demanded by the tortuous anatomy. For example, catheters must be fairly stiff at their proximal end to allow the pushing and manipulation of the catheter as it progresses through the body, and yet must be sufficiently flexible at the distal end to allow passage of the catheter tip through the loops and smaller blood vessels. Regardless, the catheter does not cause significant trauma to the blood vessel or to the surrounding tissue.

Additionally, many interventional catheters use a low friction liner on the innermost surface of the catheter to allow for the smooth passage of other interventional devices or biologic agents through the catheter. PTFE liners are one of the most lubricious and durable low-friction liners available. While PTFE offers a smooth, durable, and low friction surface, it is relatively stiff and imparts a significant added stiffness to the overall catheter construction. Often the added stiffness creates challenges and problems for the catheter in terms of reduced bendability and reduced navigability, especially at the distal end of the catheter, which needs to encounter more aggressive anatomical curvature. Previously, attempts were made to “soften” the inner liner using mechanical manipulations (i.e., axial or biaxial stretching, fatiguing, expanding, etc., but without significant effect. PTFE is extremely durable and practically inert from a polymer chemistry perspective, and attempts to soften the PTFE through chemical changes or polymer chemistry changes have not been successful. Designers must choose whether to utilize a PTFE liner that runs continuously through the distal region of the catheter (i.e., runs from proximal shaft to distal tip) and accept the limitations of reduced navigation and reduced ability to access distal anatomic territories. Alternatively, designers must terminate the PTFE liner proximal to the distal tip and replace it with something more flexible that permits easier navigation. Currently, most of the currently available neuro-interventional access catheters utilize PTFE liners that run from the proximal shaft in the distal direction and terminate around 10 cm to 20 cm from the distal tip of the catheter; in this location, the PTFE liner usually “transitions” to a new liner, often made of polyolefin, ePTFE, or another softer polymeric material. This new polymer is softer than the PTFE and thus allows for better bending ability and improved navigability. However, this softer liner is also less durable and has a less favorable frictional surface. Therefore, interventional devices being delivered through the ID of the catheter (i.e., metal stents, coils, etc.) can damage the liner during both delivery and retrieval back through the catheter, causing damage to the entire catheter shaft and resulting in the catheter failing and needing replacement during a procedure. Additionally, the transition of the two materials is not a polymeric fusing, the way other catheter transitions would be, because PTFE is a thermoset material and therefore does not melt and reflow the way conventional thermoplastics do. Therefore, the transition region can sometimes delaminate and cause further negative issues and events.

FIG. 1 illustrates a traditional catheter construction and shows sectional views of a catheter section 10 that can be constructed on an inner mandrel or core 12 that is later removed. The traditional catheter construction includes a layer 14, such as PTFE, that provides a lubricious surface for the interior of the catheter while also supporting various structural components to provide varying sections 16 and 18 of the catheter 10. This structure is often referred to as an inner liner 14. In the illustrated example, the inner liner includes a reinforced section 16 in which a braid or coil 20 (or both) is wrapped around the second layer 14. Many catheters use metal braids in the proximal end of the catheter and metal coils in the distal end of the catheters (or one under the other). The catheter of FIG. 1 is shown with the outer layers terminating at section 18 for purposes of showing the inner liner 14 and braid/coil 20.

As shown in FIG. 1, various a series of adjacently placed polymer jackets 22, 24, 26 are placed over the reinforcement layer and fused into place (such as by heating and reflowing the polymer onto the braid or coil). Different polymer durometers (i.e., “stiffness”) are used for different sections. As a result, each of these sections of catheter will have unique structural characteristics/properties, where the structural properties can include but are not limited to stiffnesses, resistance to twisting or torsion, flexibility, column strength, etc. The illustrated construction 10 provides for varying structural characteristics over the varying regions of the catheter.

In many cases, catheters must be advanced through tortuous anatomy with a decreasing vessel diameter. In such cases, the diameter of the catheter is significantly reduced such that the structural properties of the inner liner 14 adversely affect the flexibility of the catheter. Some manufacturers address these adverse issues by simply terminating the inner liner 14 prior to the distal end of the catheter. However, such designs produce catheters with reduced lubricity at the distal section, which can cause other adverse effects when trying to use the catheter to perform procedures at the distal end.

Therefore, there remains a need for a catheter having improved inner liners.

### SUMMARY OF THE INVENTION

The present disclosure includes catheters and similar medical devices having inner liners where the polymeric inner liner tubing can have regions of customized properties created by slot-type patterns in the inner liner catheter. Variations of the devices can include constructions having a catheter shaft having an axial length, the catheter shaft including an inner liner, a reinforcement structure that is exterior to the inner liner, and a tubular outer layer extending over the reinforcement structure, and the inner liner includes at least one spiral slot pattern included of a plurality of slots between a plurality of interruptions where a wall of the inner liner remains intact, wherein each slot of the plurality of slots has a length measured between each adjacent interruption.

Variations of the devices herein include catheter constructions wherein the plurality of slots includes a first pattern of slots, each having a first length and a second pattern of slots, each having a second length, where the first length and second length are different. In some variations, a pitch of the at least one spiral slot pattern varies over an axial length of the inner liner.

In some aspects, the devices herein include catheter constructions with a catheter shaft having an axial length, the catheter shaft including an inner liner, a reinforcement structure that is exterior to the inner liner, and a tubular outer layer extending over the reinforcement structure; and wherein the inner liner includes a plurality of regions extending sequentially lengthwise along the inner liner having a plurality of slots, each of the plurality of regions having a number of slots of the plurality of slots extending spirally about the inner liner; and the plurality of regions including at least a first-end region and a second-end region on opposite ends of the plurality of regions, wherein the number of slots in the first-end region is greater than the number of slots in each of a remainder of the plurality of regions such that the first-end region includes a first stiffness, wherein the number of slots in the second-end region is less than the number of slots in each of the remainder of the plurality of regions such that the second-end region includes a second stiffness, wherein the first stiffness less than the second stiffness.

In additional variations, the inner liner includes a plurality of regions extending sequentially lengthwise along the inner liner having a plurality of slots, each of the plurality of regions having a number of slots of the plurality of slots extending spirally about the inner liner; and the plurality of regions including at least a first-end region and a second-end region on opposite ends of the plurality of regions, wherein the number of slots in the first-end region is greater than the number of slots in each of the remainder of the plurality of regions such that the first-end region includes a first stiffness, wherein the number of slots in the second-end region is less than the number of slots in each of the remainder of the plurality of regions such that the second-end region includes a second stiffness, wherein the first stiffness less than the second stiffness.

In some aspects, catheter constructions can include the plurality of regions having an intermediate region between the first-end region and second-end region, where the number of slots in the intermediate region is greater than the number of slots in the second-end region and less than the number of slots in the first-end region, such that an intermediate stiffness of the intermediate region is less than the first stiffness and greater than the second stiffness.

Variations of the devices described herein relate to a catheter construction, wherein the plurality of regions includes a plurality of intermediate regions sequentially located between the first-end region and second-end region, where the number of slots in each of the plurality of intermediate regions decreases from the first-end region to the second-end region, such that a stiffness of each of the plurality of intermediate regions increases towards the second-end region.

In additional variations, a pitch of the number of slots in the first-end region increases in a direction away from the second-end region, such that the first stiffness of the first-end region decreases as a pitch decreases.

The slots can extend partially or fully through a wall of the inner liner.

The present disclosure also includes catheter constructions having a catheter shaft having an axial length, the catheter shaft including an outer layer and an inner liner located coaxially within the outer layer, the inner liner having a lumen extending therethrough, a proximal end, and a distal end; and wherein the inner liner includes one or more slots extending helically along the inner liner, where a depth of the one or more slots varies along an axial length of the catheter shaft.

The one or more slots can include a plurality of slots between a plurality of interruptions where a wall of the inner liner remains intact, wherein each slot has a depth that extends from the wall of the inner liner towards the lumen of the inner liner in a radial direction.

In some variations, the depth of the one or more slots improves preferential bending of the inner liner. In additional variations, the depth of the one or more slots gradually increases from the proximal end of the inner liner to the distal end of the inner liner.

In some aspects, the techniques described herein relate to a catheter construction, wherein the inner liner includes a plurality of regions extending sequentially lengthwise along the inner liner, wherein a number of slots in each of the plurality of regions vary.

Variations of the devices include a catheter construction, wherein the plurality of regions includes a first region and a second region, wherein the depth of the one or more slots in the first region is greater than the depth of the one or more slots in the second region.

Variations of the devices can include slots where the depth of the one or more slots in a first region of the inner liner extends from a surface of the inner liner through to the lumen. Additionally, in a second region, the depth of the one or more slots decreases such that the depth extends from the surface of the inner liner and partially through a wall of the inner liner.

The devices described herein can include a reinforcement structure exterior to the inner liner and within the outer layer extending over the reinforcement structure.

In some aspects, the techniques described herein relate to a catheter construction, wherein the inner liner includes a first radial side and a second radial side opposite to the first radial side, the inner liner further including a first segment and a second segment, wherein at the first segment the depth of the one or more slots at the first radial side is less than the depth of the one or more slots on the second radial side.

In some aspects, the techniques described herein relate to a liner for use with a medical tubing, the liner including a liner body extending along an axial length and including a lumen extending therethrough a proximal end and a distal end; and wherein the liner body includes one or more slots extending helically along the liner body, where a depth of the one or more slots vary along the axial length.

In some aspects, the techniques described herein relate to a liner, wherein the one or more slots include a plurality of slots between a plurality of interruptions where a wall of the liner body remains intact, wherein each slot has a depth that extends from the wall of the liner body towards the lumen of the liner body in a radial direction.

The devices of the present disclosure allow for a considerable number of combinations and permutations of different variations of liners and catheters, as well as a combination of aspects of those structures as well. It is contemplated that any of the requirements and elements described herein can be combined with any independent claim where the requirements of the independent claims would not contradict the various elements.

### BRIEF DESCRIPTION OF THE FIGURES

FIG. 1 illustrates a traditional catheter construction and shows sectional views of a catheter section that is constructed on an inner extruded tube.

FIG. 2 shows a partial sectional view of an improved catheter incorporating an improved polymeric liner.

FIG. 3A illustrates a variation of a modified liner.

FIGS. 3B and 3D show magnified views of regions 3B and 3C, respectively, in FIG. 3A.

FIG. 3C shows an alternate construction of a pattern similar to FIG. 3B.

FIG. 4A illustrates another variation of a modified liner.

FIG. 4B shows magnified a view of region 4B in FIG. 4A.

FIGS. 5A and 5B illustrate an additional aspect of the modified layers where a depth of slots in the liner can vary along the liner.

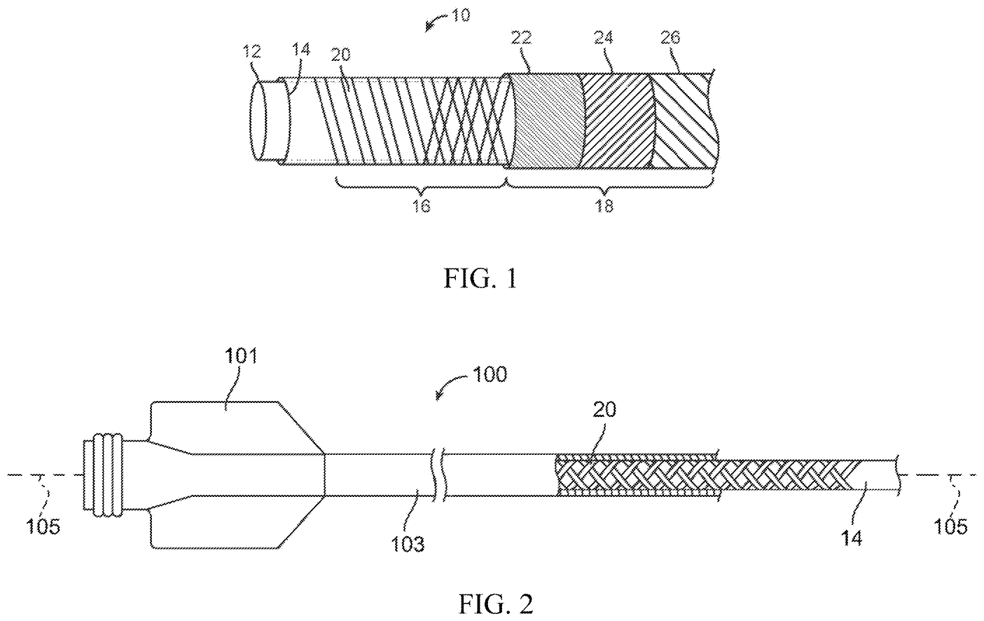

### DETAILED DESCRIPTION

The catheter configuration discussed herein can be used in a variety of devices where different regions are selected for customized properties. The configurations described herein can be incorporated into various medical devices or can be used as catheter shafts. Furthermore, in some variations, the construction features of the present disclosure are not limited to in-dwelling medical devices and can be used for any device requiring tubing.

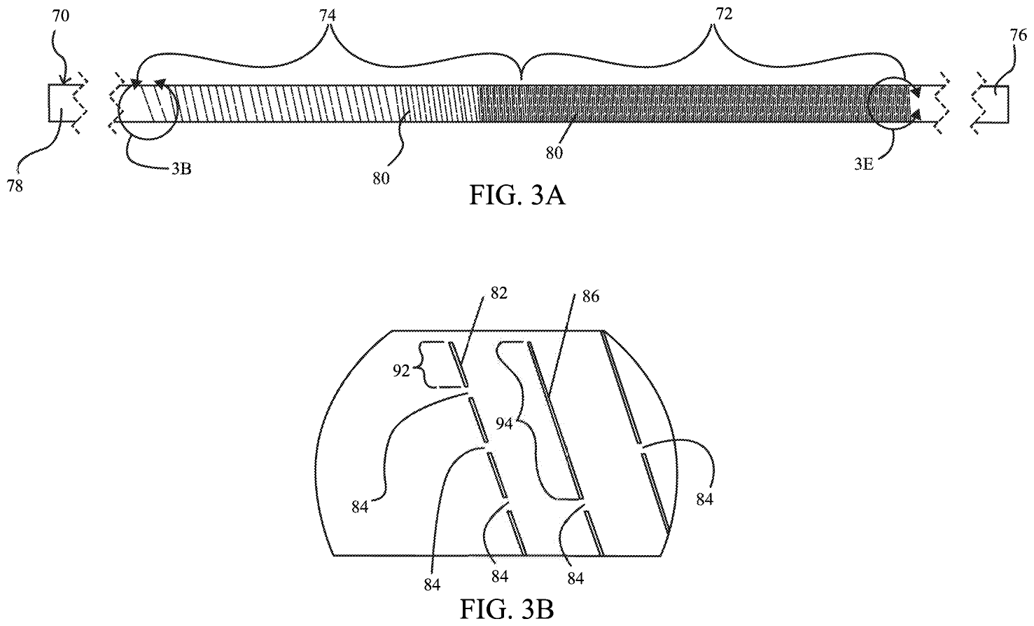

FIG. 2 shows a partial sectional view of an improved catheter 100 incorporating an improved inner liner 14, as discussed herein. The catheter construction discussed herein can incorporate any number of features known by those skilled in the art of catheter construction. Such features are omitted herein so that the focus of the improved catheter polymeric inner liner 14 can be explained. Furthermore, the improved catheter construction disclosed herein can be incorporated into any number of catheters that can benefit from the customization of features provided by the improved construction. For example, such catheters include but are not limited to, distal access catheters, sheaths, guide catheters, balloon catheters, intracranial support catheters, microcatheters, arterial line catheters, central venous catheters, pulmonary artery catheters, coronary and cardiac catheters, and peripheral catheters, etc. Additional variations of the improved construction can be used in any polymeric tubular structure.

As shown, in one variation of the device, the tubular construction or shaft of the catheter 100 extends from a hub 101 and can be formed by the improved outer polymeric layer 103, discussed below, that overlays a braid 20, coil or other support structure commonly used with catheters. The braid 20 is positioned about a tubular inner liner 14 (commonly constructed from PTFE, but other materials are within the scope of this disclosure). As shown in FIG. 2, a polymeric layer 103, is usually the outermost component of the catheter tubing.

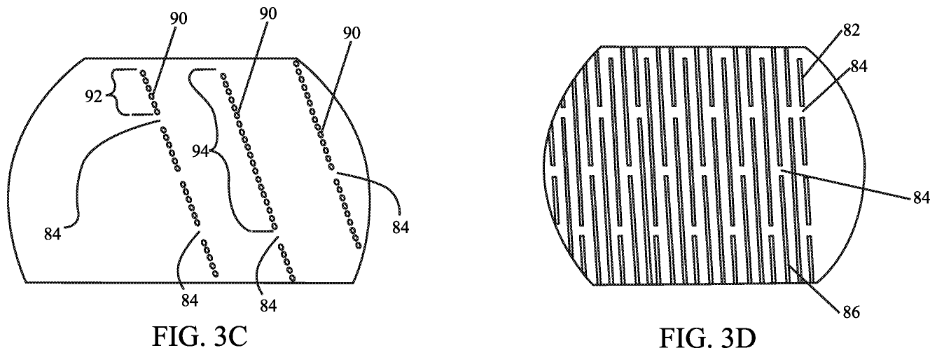

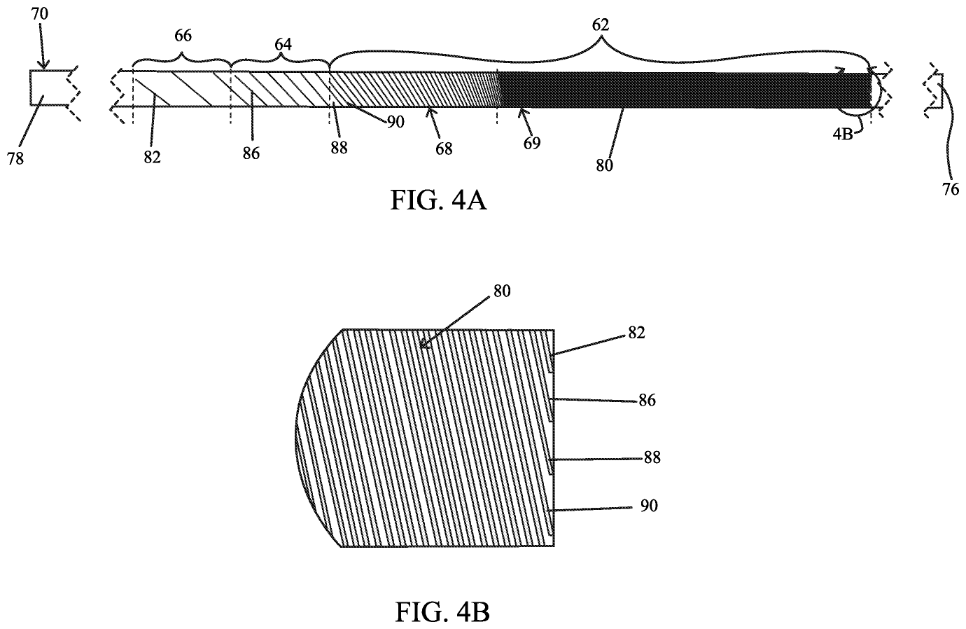

FIGS. 3A to 4B illustrate another aspect of improved devices under the present disclosure that can vary the structural characteristics of an inner liner of a catheter or other medical device, especially at a distal tip of the catheter/medical device. The present disclosure includes the use of such a novel liner in any portion of the catheter or device, and the construction is not limited to an inner liner.

Traditional catheter designs include the use of a lubricious liner within the catheter (see, e.g., liner 14 in FIG. 2). Typically, the liner is fabricated from a polytetrafluoroethylene (PTFE) material. However, any liner material can be modified as described herein to remain within the scope of this disclosure. In certain catheters, the PTFE liner adds stiffness to the catheter, especially small-diameter catheters or small-diameter regions. In some conventional catheters, the PTFE liner is terminated to prevent the stiffness added by the PTFE liner from impacting the catheter performance. In alternative variations, a distal section of the PTFE liner is replaced with a softer, more compliant liner material. FIG. 3A illustrates a modified variation of a liner 70 for use as an inner liner in a catheter (see, e.g., element 14 of FIG. 3A). The liner 70 can be modified to produce varying structural characteristics over regions of the inner liner 70. The variation shown includes slots that can extend through an entirety of a wall of the liner, can extend partially, or a combination of both.

FIG. 3A shows a flat cut pattern of a liner 70, having at least a first region 72 and a second region 74 that is modified to increase flexibility of the liner 70. However, any number of regions is within the scope of this disclosure. The liner 70 includes a spiral slot pattern 80 comprising slots that extend through a wall of the liner 70. In the example shown in FIG. 4A, the liner includes a first region 72, where the slot pattern 80 comprises a constant pitch. The second region, 74, includes a variable pitch. In the illustrated example, the liner 70 includes a distal region 76 and proximal region 78, where the slot pattern 80 is terminated before reaching the respective regions 76, 78. However, any number of variations where the slot pattern extends into such regions are within the scope of this disclosure

FIGS. 3B and 3D show magnified views of areas 3B and 3D from FIG. 3A. FIG. 3B illustrates that slots 82 of the slot pattern 80 can be interrupted by region 84 of the liner wall. FIG. 3B shows a slot 82 as having 4 interrupting regions 84. In this example, the first four slots 82 include 4 interruptions 84 that occur every 90 degrees over the circumference of the liner. The next slot, 86, includes an interruption every 270 degrees over the circumference of the liner. As noted above, slots 82 86 are formed with a variable pitch over region 74.

FIG. 3B also illustrates that a length of the slots 82 86 can be measured between each adjacent interruption. As shown in FIG. 3B, the slot pattern 80 includes four slots 82 of equal length 92 followed by slots 86 having a larger length 94. The lengths of the slots can vary along the entirety of the slot pattern 80 or along various regions 72, 74 to produce desired stiffness characteristics of the liner 70.

FIG. 3C illustrates an alternate configuration where the slots are in a similar configuration to that of FIG. 3B, but in FIG. 3C, the slots comprise a plurality of holes or openings 90 as opposed to the continuous slots shown in FIG. 3B. Similar to FIG. 3B, the pattern can comprise a series of opening 90 having a consistent length 92 followed by a series of openings 90 having a longer length 94. In one example, a slot formed from a series of openings will be considered to extend between the interrupting region 84 of the liner wall. Variations of the liner can include continuous slots as well as slots formed from the plurality of openings on the same liner. Any variation disclosed herein can include slots formed from a series of openings. Doing so can maintain a continuity of the liner.

FIG. 3D illustrates a magnified view of slot pattern 80 having a constant pitch. As shown, the last 4 slots 82 include 54 interruptions 84 every 90 degrees over the circumference of the liner. Slots 86 include an interruption every 270 degrees over the circumference of the liner.

FIG. 4A illustrates another flat cut pattern to demonstrate a slot pattern 80 in a variation of a liner 70 as described above. In this example, the liner 70 includes a distal section 76 and proximal section without any slot pattern 80. However, variations of the liners can include slot patterns that extend to either/both regions. The slot pattern 80 varies over a number of regions 62, 64, 66 of the liner 70. The number of regions shown and the number of slots 82, 86, 88, 90 are for illustrative purposes only. Any number of regions and slots are within the concept of this disclosure. As shown, the plurality of regions 62, 64, and 66 extend sequentially lengthwise along the liner 70 (e.g., 62 left-most, 66 right-most, and 64 intermediate). Each region includes a number of slots 82, 86, 88, 90, extending spirally about the liner 70. The liner 70 includes end regions 62 and 66 on opposite ends of the plurality of regions. In the illustrated example, the number of slots in the first-end region 62 is greater than the number of slots in each of the other plurality of regions 64 and 66 such that the first-end region 62 comprises a first stiffness. The number of slots in the second-end region 66 is less than the number of slots in each of the other plurality of regions 62 and 64 such that the second-end region 66 comprises a second stiffness, wherein the first stiffness less than the second stiffness.

Laser cut patterns can be formed with a single spiral pattern or multiple spiral patterns adjacent to one another. In the example shown in FIG. 4A, this pattern is formed from 4 individual spiral patterns (similar to a coil formed from multiple wires, what is commonly referred to as a “multi-filar” coil, or in this case, a multi-filar spiral cut pattern). Utilizing multiple spiral patterns to make the overall pattern produces a region that is stronger and has greater mechanical integrity than a single spiral would produce. In this example, the 4 individual spiral patterns all terminate at different locations proximally to further enhance the gradualness of the transition from cut region to uncut region.

FIG. 4A illustrates the plurality of regions includes an intermediate region between the first-end region and second-end region, where the number of slots in the at least one intermediate region is greater than the number of slots in the second-end region and less than the number of slots in the first-end region, such that an intermediate stiffness of the intermediate region is less than the first stiffness and greater than the second stiffness.

FIG. 4A also shows an intermediate region 64 located between the first-end region 62 and second-end region 64. Again, additional variations of the device include any number of intermediate regions that can be located sequentially between the end regions, where the number of slots in each intermediate region decreases from the first-end region 62 to the second-end region 66, such that a stiffness of each of the plurality of intermediate regions increases towards the second-end region 66. In the illustrated example, the stiffness of the liner 70 declines towards the right. In most variations, the stiffness of the liner will decline towards a distal end of the finished catheter. However, variations of the device are not limited to such a configuration.

Turning to the specifics of FIGS. 4A and 4B, a single slot 82 extends through a wall of the liner 70 in a helical or spiral pattern in the second-end region 66. This slot spirals around the liner 70 for 2 revolutions until the intermediate region 64 begins. In this region, where a second slot 86 begins. The intermediate region 64 allows for approximately 3 revolutions before the first-end region 62, in which a third slot 88 and a fourth slot 90 begin. FIG. 4A also shows that the first-end region 62 maintains four slots 82, 86, 88, and 80, but the pitch of the slots 82, 86, 88, and 80 varies in region 62. In the illustrated example, a pitch of the number of slots in section 68 of end region 62 is lower than section 69, such that the stiffness of section 68 is less than that of 69. The pitch of the slot pattern 80 can change to a constant pitch in section 69 until the slots end. In the illustrated variation, there are approximately 10 rotations before the start of the constant pitch section 66. FIG. 14B illustrates a magnified view of region 14B from FIG. 4A, where the slots 82, 86, 88, 90 terminate at 90-degree rotational intervals. The construction shown increases flexibility of the liner 70 in an increasing manner from region 66 to region 62. As shown, the slots 82, 86, 88, 90 can terminate 90 degrees rotationally about the circumference of the liner 70 from one another. Additional variations of liners can include any number of slots as well as any number of regions. Furthermore, the slots pattern 80 of FIG. 4A can include interruptions, as shown in FIG. 3A. It is noted that the modification of liners can include liners fabricated from any material beyond PTFE.

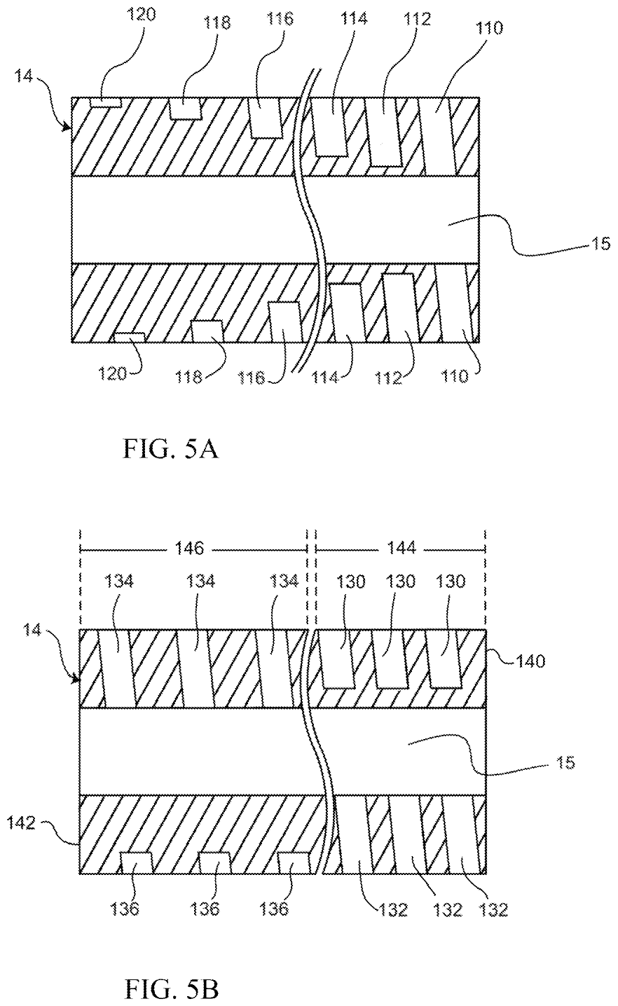

FIG. 5A illustrates a cross-sectional view of a liner 14 having one or more slots 110-120 helically extending along the liner 14 as disclosed above. However, one additional aspect of the improved liner 14 is that the slots 110-120 can vary in depth. As shown, some slots 110 can extend through the wall of the liner 14 from an exterior surface to an internal lumen 15 of the liner 15. A further additional aspect of an improved liner 14 is that the slots 110-120 can vary in depth along a length of the liner 14. As shown, the slots of the liner variation 14 in FIG. 5A are deepest, starting at slot 110. The depth of the slots decrease towards slot 120. The depth of the slots can continuously vary, as shown in FIG. 5A. Alternatively, the depth of slots can vary stepwise such that a first region of slots has a set depth, then an adjacent group of slots has a different depth. In one variation, the depth of the slots is greatest at a distal region of the liner to provide increased flexibility at the distal end, and the depth decreases towards a proximal end of the liner. Alternatively, variations of the device include a liner where the depth of the slots increase in a proximal to distal direction (i.e., the deepest slots are towards a proximal portion of a liner).

FIG. 5B illustrates another variation where the depth of the slots 130-136 is not uniform about the circumference of the liner 14. As shown in FIG. 5B, slots 130 can be a fixed depth while the corresponding slots 132, located 180 degrees opposite, comprise a different depth. As shown in FIG. 5B, slots 130 do not extend to a lumen 15 of the liner 14, while the 180-degree opposing slots 132 extend through the wall to the lumen 15. Likewise, another portion of the liner 14 can include slots 136 of a shallow depth while the slots 134, arranged 180 degrees opposite, extend to a different depth (in this case, through the wall to the lumen 15). Selectively controlling the depth of slots on specific sides and regions of the liner 14 allows for customizing a preferential directional bend of the liner.

To further illustrate the concept of altering the depth in the circumferentially extending slot, FIG. 5B illustrates a liner 14 as having a first radial side 140 and a second radial side 142. These radial sides are simply opposing sides of the liner 14, which will have a circular, oval cross, or polygonal sectional shape. The liner 14 can also have two or more segments 144, 146, where preferential bending is different for each segment. In such a case, the liner 14 shown includes slots 130 in the first radial side 140 of the first segment 144, which are shallower than slots 132 in the second radial side 142 of the first segment. Clearly, the change in depth can be incremental or stepwise. Furthermore, the radial sides are not required to be opposite and can be two radial spans of the liner body. The slot 130 can be a single slot that continuously spirals about the liner 14. Alternatively, each slot 130 132 can be a series of circumferential slots that are separated from the adjacent slot. FIG. 5B also shows the second segment 146, where the depth of the one or more slots 134 at the first radial side 140 is greater than the depth of the one or more slots 136 on the second radial side 142. Again, the slots for the variations shown in FIGS. 5A and 5B can be one or more continuous slots that spirally extend, or they can be a series of slots that are adjacent to one another.

As for other details of the present invention, materials and manufacturing techniques may be employed as within the level of those with skill in the relevant art. The same may hold true with respect to method-based aspects of the invention in terms of additional acts that are commonly or logically employed. In addition, though the invention has been described in reference to several examples, optionally incorporating various features, the invention is not to be limited to that which is described or indicated as contemplated with respect to each variation of the invention.

Various changes may be made to the invention described, and equivalents (whether recited herein or not included for the sake of some brevity) may be substituted without departing from the true spirit and scope of the invention. Also, any optional feature of the inventive variations may be set forth and claimed independently or in combination with any one or more of the features described herein. Accordingly, the invention contemplates combinations of various aspects of the embodiments or combinations of the embodiments themselves, where possible. Reference to a singular item, includes the possibility that there are plural of the same items present. More specifically, as used herein and in the appended claims, the singular forms “a,” “and,” “said,” and “the” include plural references unless the context clearly dictates otherwise.

It is important to note that where possible, aspects of the various described embodiments, or the embodiments themselves can be combined. Where such combinations are intended to be within the scope of this disclosure.

We claim:

1. A catheter construction comprising:
a catheter shaft having an axial length, the catheter shaft comprising an inner liner, a reinforcement structure that is exterior to the inner liner, and a tubular outer layer extending over the reinforcement structure; and
the inner liner comprises at least one spiral slot pattern comprised of a plurality of slots between a plurality of interruptions where a wall of the inner liner remains intact, wherein each slot of the plurality of slots has a length measured between each adjacent interruption.

2. The catheter construction of claim 1, wherein the plurality of slots comprises a first pattern of slots each having a first length and a second pattern of slots each having a second length, where the first length and second length are different.

3. The catheter construction of claim 1, wherein a pitch of the at least one spiral slot pattern varies over an axial length of the inner liner.

4. A catheter construction comprising:
a catheter shaft having an axial length, the catheter shaft comprising an inner liner, a reinforcement structure that is exterior to the inner liner, and a tubular outer layer extending over the reinforcement structure; and
wherein the inner liner comprises a plurality of regions extending sequentially lengthwise along the inner liner having a plurality of slots, each of the plurality of regions having a number of slots of the plurality of slots extending spirally about the inner liner; and
the plurality of regions including at least a first-end region and a second-end region on opposite ends of the plurality of regions, wherein the number of slots in the first-end region is greater than the number of slots in each of a remainder of the plurality of regions such that the first-end region comprises a first stiffness, wherein the number of slots in the second-end region is less than the number of slots in each of the remainder of the plurality of regions such that the second-end region comprises a second stiffness, wherein the first stiffness less than the second stiffness.

5. The catheter construction of claim 4, wherein the inner liner comprises a plurality of regions extending sequentially lengthwise along the inner liner having a plurality of slots, each of the plurality of regions having a number of slots from the plurality of slots extending spirally about the inner liner; and
the plurality of regions including at least a first-end region and a second-end region on opposite ends of the plurality of regions, wherein the number of slots in the first-end region is greater than the number of slots in each of the remainder of the plurality of regions such that the first-end region comprises a first stiffness, wherein the number of slots in the second-end region is less than the number of slots in each of the remainder of the plurality of regions such that the second-end region comprises a second stiffness, wherein the first stiffness less than the second stiffness.

6. The catheter construction of claim 4, wherein the plurality of regions includes an intermediate region between the first-end region and second-end region, where the number of slots in the intermediate region is greater than the number of slots in the second-end region and less than the number of slots in the first-end region, such that an intermediate stiffness of the intermediate region is less than the first stiffness and greater than the second stiffness.

7. The catheter construction of claim 4, wherein the plurality of regions includes a plurality of intermediate regions sequentially located between the first-end region and second-end region, where the number of slots in each of the plurality of intermediate regions decreases from the first-end region to the second-end region, such that a stiffness of each of the plurality of intermediate regions increases towards the second-end region.

8. The catheter construction of claim 4, wherein a pitch of the number of slots in the first-end region increases in a direction away from the second-end region, such that the first stiffness of the first-end region decreases as a pitch decreases.

9. The catheter construction of claim 4, where at least one slot of the plurality of slots extends through a wall of the inner liner.

10. The catheter construction of claim 4, wherein at least one slot of the plurality of slots comprises a plurality of adjacent openings.

11. A catheter construction comprising:
a catheter shaft having an axial length, the catheter shaft comprising an outer layer and an inner liner located coaxially adjacent to the outer layer, the inner liner having a lumen extending therethrough, a proximal end, and a distal end; and
wherein the inner liner comprises one or more slots extending helically along the inner liner, where a depth of the one or more slots vary along an axial length of the catheter shaft.

12. The catheter construction of claim 11, wherein the one or more slots comprises a plurality of slots between a plurality of interruptions where a wall of the inner liner remains intact, wherein each slot has a depth that extends from the wall of the inner liner towards the lumen of the inner liner in a radial direction.

13. The catheter construction of claim 11, wherein the depth of the one or more slots improves a transitional bending of the inner liner.

14. The catheter construction of claim 11, wherein the depth of the one or more slots gradually increases from the proximal end of the inner liner to the distal end of the inner liner.

15. The catheter construction of claim 11, wherein the inner liner comprises a plurality of regions extending sequentially lengthwise along the inner liner, wherein a number of slots in each of the plurality of regions varies.

16. The catheter construction of claim 15, wherein the plurality of regions comprises a first region and a second region, wherein depth of the one or more slots in the first region is greater than the depth of the one or more slots in the second region.

17. The catheter construction of claim 11, wherein the depth of the one or more slots in a first region of the inner liner extends from a surface of the inner liner through to the lumen.

18. The catheter construction of claim 17, wherein in a second region the depth of the one or more slots decrease such that the depth extends from the surface of the inner liner and partially through a wall of the inner liner.

19. The catheter construction of claim 11, further comprising a reinforcement structure exterior to the inner liner and within the outer layer extending over the reinforcement structure.

20. The catheter construction of claim 11, wherein the inner liner comprises a first radial side and a second radial side opposite to the first radial side, the inner liner further comprising a first segment and a second segment, wherein at the first segment the depth of the one or more slots at the first radial side is less than the depth of the one or more slots on the second radial side.

21. The catheter construction of claim 20, wherein at the second segment the depth of the one or more slots at the first radial side is greater than the depth of the one or more slots on the second radial side.

22. A liner for use with a medical tubing, the liner comprising:
a liner body extending along an axial length and comprising a lumen extending therethrough, a proximal end, and a distal end; and
wherein the liner body comprises one or more slots extending helically along the liner body, where a depth of the one or more slots vary along the axial length.

23. The liner of claim 22, wherein the one or more slots comprises a plurality of slots between a plurality of interruptions where a wall of the liner body remains intact, wherein each slot has a depth that extends from the wall of the liner body towards the lumen of the liner body in a radial direction.

24. The liner of claim 22, wherein the depth of the one or more slots improves preferential bending of the liner.

25. The liner of claim 22, wherein the depth of the one or more slots gradually increases from the proximal end of the liner body to the distal end of the liner body.

26. The liner of claim 22, wherein the liner body comprises a plurality of regions extending sequentially lengthwise along the liner body, wherein a number of slots in each of the plurality of regions varies.

27. The liner of claim 26, wherein the plurality of regions comprises a first region and a second region, wherein depth of the one or more slots in the first region is greater than the depth of the one or more slots in the second region.

28. The liner of claim 22, wherein the depth of the one or more slots in a first region of the liner extends from a surface of the liner through to the lumen.

29. The liner of claim 28, wherein in a second region the depth of the one or more slots decreases such that the depth extends from the surface of the liner and partially through a wall of the liner.

30. The liner of claim 22, wherein at least one slot of one or more slots comprises a plurality of adjacent openings.
